(19) United States
(12) Patent Application Publication
(10) Pub. No.: US 2016/0058659 A1
(43) Pub. Date: Mar. 3, 2016

(54) PERSONAL GROOMING DEVICE

(71) Applicant: Anton ANGELOV, Bartlett, IL (US)

(72) Inventor: Anton ANGELOV, Bartlett, IL (US)

(21) Appl. No.: 14/844,467

(22) Filed: Sep. 3, 2015

Related U.S. Application Data
(60) Provisional application No. 62/045,378, filed on Sep. 3, 2014.

(51) Int. Cl. A61H 23/02; A61H 23/00; A45D 29/14; A61B 17/54; A45D 29/05; A45D 29/00

(52) U.S. Cl. CPC A61H 23/0263; A45D 29/05; A45D 29/007; A45D 29/14; A61B 17/54; A61H 23/004

(57) ABSTRACT

A personal grooming device for caring for finger nails, toe nails, skin and hair. The personal grooming device includes a handle enclosing a motor and a power supply. The handle connects to an orbital vibrating mechanism powered by the motor. The orbital vibrating mechanism includes a mounting plate for attaching a various tools including a nail file, an exfoliator, a callous remover, a massager, and/or a brush. The various tools can be attached to and removed from the mounting plate repeatedly allowing for the personal grooming device to accommodate a variety of personal grooming needs.

### CROSS REFERENCE TO RELATED APPLICATION

This application claims the benefit of Provisional U.S. Patent Application No. 62/045,378, filed on 3 Sep. 2014. The Provisional U.S. patent application is hereby incorporated by reference herein in its entirety and is made a part hereof, including but not limited to those portions which specifically appear hereinafter.

### FIELD OF THE INVENTION

This invention is directed to a personal grooming device and more particularly to an electrically powered device with changeable tools for caring for finger nails, toe nails, skin, and hair.

### BACKGROUND OF THE INVENTION

General personal care requires multiple tools to file nails, buff nails, remove polish, and exfoliate skin. In general, the tools used for such skin and nail care are single-purpose mechanical tools. In addition to being single-purpose tools, these mechanical tools require time and only provide merely satisfactory results.

As such, there is a need for an improved personal grooming device that can be used for multiple purposes and provides rapid and superior results to traditional tools.

### SUMMARY OF THE INVENTION

The invention is directed to a grooming device for caring for finger nails, toe nails, skin, and hair.

In an embodiment of this invention, the grooming tool comprises an electric nail buffer/file intended to replace existing traditional nail files. The subject invention is practical and much faster than a traditional nail file and provides better results. Additionally, other attachments may be connected to the grooming tool for other personal needs including exfoliating skin, removing callouses and/or to care for hair.

In a preferred embodiment, the grooming device includes an elongated handle, housing a motor and power supply, and a swing head with a mounting plate connectable to the motor to provide orbital motion to a tool. The swing head is preferably connectable to a variety of tools to care for finger nails, toe nails, skin and hair. For example, the tools may include a filing tool, a buffing tool, an exfoliating tool, a callous removal tool, a massage head, and a brush.

In a preferred embodiment, the handle and the swing head are manufactured with plastic and/or metal materials. However, any light-weight and durable material may be used. The handle can have any desirable form, shape, size and/or color. Preferably, the handle comprises an ergonomic shape that allows a user to comfortably use the grooming device. The handle may further include rubber grips or inserts to comfortably handle the grooming device.

The handle preferably includes a hollow interior to accommodate a power supply and a motor capable of providing orbital or other vibratory movement to the tool. The motor preferably comprises an electrical motor with a small drive shaft extending from the motor and connectable to an orbital vibrating mechanism in the swing head. Alternatively, the grooming device may include a mechanical vibrator, such a spring-driven vibratory mechanism. In a preferred embodiment, the power supply comprises rechargeable batteries. However, other power supplies may be used including a corded power supply connectable to an electrical outlet.

The grooming device may further include an on/off switch, a function switch and/or an indicator light to indicate, for example, an operating mode, a recharging mode and/or another function. The light can be of any form, size, color, material, and can be located anywhere on the handle.

According to a preferred embodiment of this invention, the swing head is removably connectable to the handle with the shaft of the motor operatively connectable to a gear shaft within the swing head. The gear shaft connects to the mounting plate with an eccentric gear providing orbital motion to the mounting plate and, in turn, to a tool attachable to the mounting plate.

In a preferred embodiment, a variety of tools are attachable to the mounting plate. For example, but not limited to filing tools, emery boards, nail polish removers, callous removers, buffing tools, exfoliators, massage heads, and brushes. In embodiments of the invention, the grooming device may include various grit to allow for shaping, filing, and buffing of finger and toe nails. The removable tools preferably attach to the mounting plate through a hook and loop mechanism. Alternatively, the grooming device may use alternative connectors for attaching the removable tools to the mounting plate.

In an embodiment of this invention, the removable tools are adjustable, preferably pitch adjustable, providing different angles for operating on the nails.

Components of the removable tools, including pads, brushes and/or replaceable heads, are all washable as is the device itself which is preferably sealed for such purpose.

In operation, the motor, connector shafts and the eccentric gear preferably impart an orbital motion on the mounting plate and the removable tool. Alternatively, the grooming device may provide a linear motion and/or a pulsating motion.

### BRIEF DESCRIPTION OF THE DRAWINGS

FIG. 1 is a perspective view of a grooming device according to one embodiment of this invention.

FIG. 2 is a partial exploded view of the grooming device shown in FIG. 1.

FIG. 3 is a side view of a portion of the grooming device according to another embodiment of this invention with a portion of a swing head shell of a swing head removed to show internal components.

FIG. 4 is a partial exploded view of the swing head shown in FIG. 3.

### DETAILED DESCRIPTION OF THE INVENTION

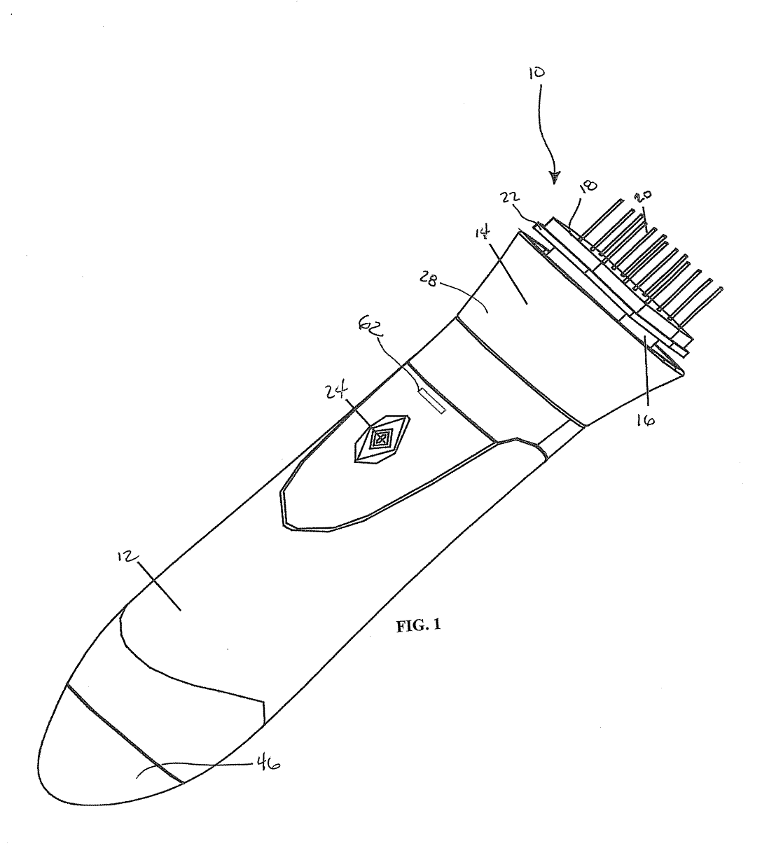

FIG. 1 shows is a perspective view of a grooming device 10 according to one embodiment of this invention, comprising a handle 12, a swing head 14, an orbital vibrating mechanism 16 for imparting an orbital motion on a tool 18. In this embodiment, the tool 18 comprises a brush 20 however, the tool may comprise any type of tool including, but not limited to, a nail file, a buffing tool, a callous remover and an exfoliator. The tool 18 is preferably attachable to and detachable from the grooming device 10 to allow a variety of tools 18 to be used with the handle 12. In a preferred embodiment, the tool 18 connects to a mounting plate 22 with a hook and loop fastener 60. However, it should be understood that other types of releasable connectors may be used.

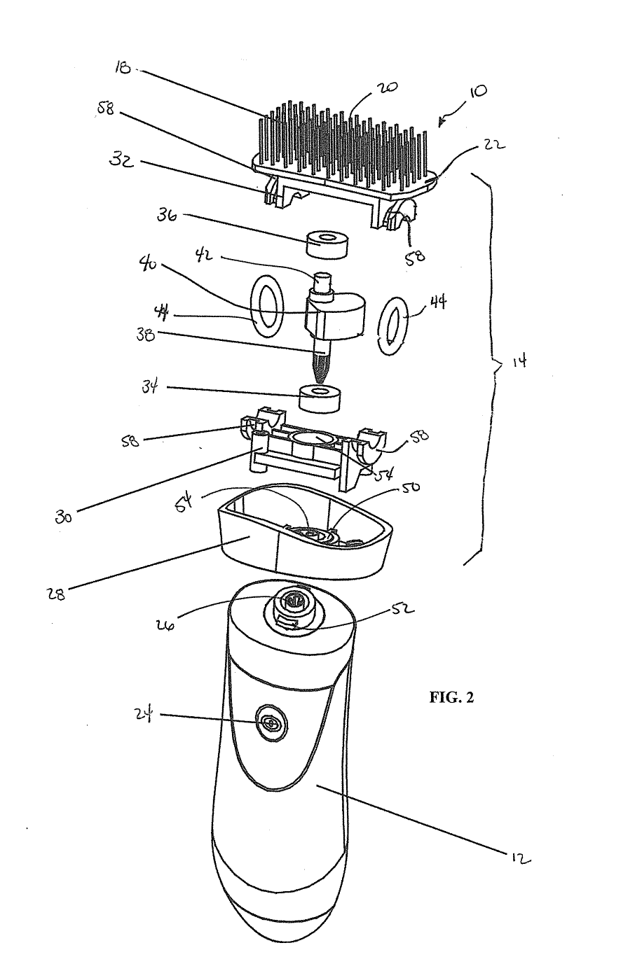

As best shown in FIGS. 1 and 2, the handle 12 comprises an elongated body enclosing an electrical motor (not shown) and a portion of a drive mechanism 26 for the tool 18. The handle 12 preferably comprises a hollow plastic body with an ergonomic shape. The handle 12 may be manufactured using techniques known to those of skill in the art, such as plastic injection molding, to have any desirable form, shape, size, and/or color. However, it should be understood that the handle may be manufactured from any material including light metals and other durable materials. The handle 12 may further include a texturized and/or non-slip surface for improved grip.

In a preferred embodiment of the invention, the handle includes a switch 24 accessible along an outer surface of the handle 12. The switch 24 comprises an electronic switching circuit for activating the electrical motor contained within the handle 12. The circuit may provide for additional controls of the electrical motor including, but not limited to speed control, automatic shut-off, and changing rotation direction. Alternatively, the handle may include additional switches or controls for controlling various features of the grooming device 10.

In a preferred embodiment, the handle 12 provides a water-resistant, sealed enclosure for the motor, power supply and the drive mechanism, allowing the grooming tool 10 to be washed without damaging the motor and other internal components. In the embodiment shown in FIG. 2, the drive mechanism 26 provides rotation motion from the electric motor while preventing water from entering the handle and possibly damaging the electric motor and power supply. In this embodiment, the swing head 14 is preferably manufactured from materials that are not damaged when exposed to or submerged in water to allow for easy cleaning.

In a preferred embodiment of this invention, the motor and drive mechanism provide a rotational motion to the swing head 14. Alternatively, the motor and drive mechanism may be designed to provide a transverse, oscillating, pulsating, or another type of motion. As discussed above, the handle 12 houses the electric motor and power supply. The handle 12 may further enclose a drive circuit, a central processing unit, and a memory for performing various functions of the grooming device 10. In the embodiment of FIG. 1, the handle 12 includes an access door 46 to provide access the power supply and/or the motor. In a preferred embodiment, the power supply is a rechargeable battery. Alternatively, the power supply may comprise disposable batteries or a cord for connection to an electrical outlet. The handle 12 preferably further includes an output unit 62 for indication operation and various functions of the grooming device 10. The output unit 62 may comprise a light (such as a light emitting diode), a speaker, a buzzer and/or a display (such as an LED or LCD display). In another embodiment, the grooming device 10 may further include a grooming light to illuminate an area being worked on, for example fingernails. In a preferred embodiment, the grooming light projects from a body of the handle 12 or swing head 14 to shine a light in proximity to an end of a tool attached to the mounting plate 22.

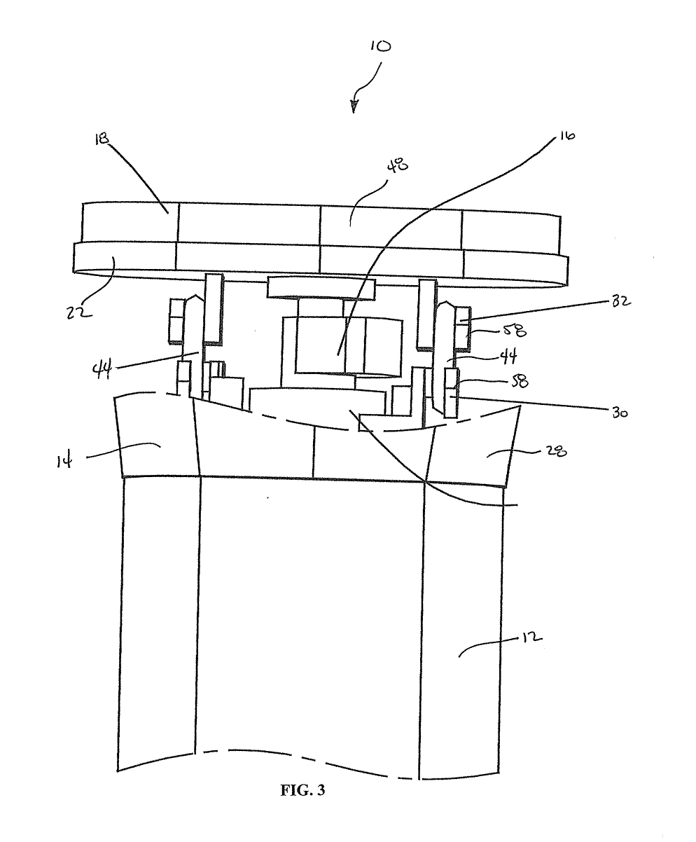

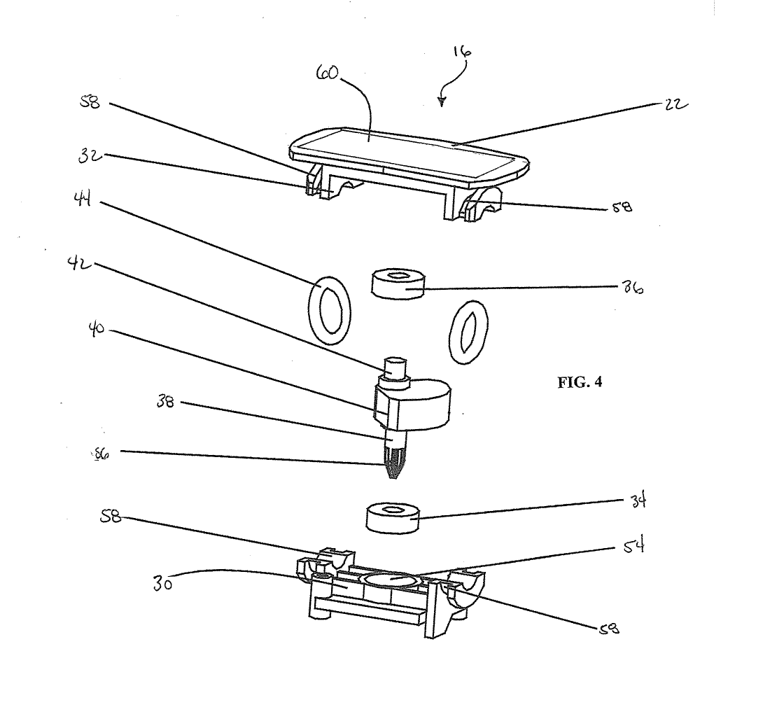

As discussed above, the grooming device of this invention can be connected to various tools. For example, FIG. 2 illustrates an assembly view of the swing head 14 of the grooming tool 10 of this invention with a brush head 20. FIG. 3 illustrates a side view of a portion of the grooming device with a portion of the swing head removed to show internal components and with an emery board 48 attached to the mounting plate 22. FIG. 4 illustrates an assembly view of the swing head without a tool attached to the mounting plate 22 showing the fastener 60.

As best shown in FIG. 2, the swing head 14 is connectable to the handle 12 with a circular slot 50 and tab 52 connector providing a releasable connection. However, it should be understood that the swing head 14 may be connected to the handle 12 with other types of releasable connectors. Alternatively, the swing head 14 and handle 12 may be formed as a unitary component.

In the embodiment of FIG. 2, the swing head 14 includes a swing head shell 28, an under fixing part 30, an upper fixing part 32, a lower bearing 34, an upper bearing 36, a gear shaft 38 with an eccentric gear 40 and an orbital shaft 42, and a plurality of o-rings 44. However, it should be understood that in other embodiments the swing head 14 may not comprise all these components and some components may be combined into a single component.

The swing head shell 28 preferably comprises an open-top housing to protect the other components of swing head 14. The swing head is preferably manufactured of plastic but may be manufactured from any other durable material including metal. The swing head 28 includes an opening 54 to allow the gear shaft 38 of the orbital vibrating mechanism 16 to connect to the drive mechanism 26 of the motor. The under fixing part 30 of the vibrating mechanism 16 is preferably connected to the swing head shell 28 with a pair of screws, however other connectors such as adhesive may be used. Alternatively, the swing head shell 28 and the under fixing part 30 may be molded as a single component.

In a preferred embodiment of this invention the orbital vibrating mechanism 16 comprises the under fixing part 30, the upper fixing part 32, the lower bearing 34, the upper bearing 36, the gear shaft 38, the eccentric gear 40, the orbital shaft 42, and the o-rings 44. IN an alternative embodiment, the orbital vibrating mechanism 16 may comprise another type of mechanism for imparting vibration including, but not limited to, motors with an off-center weight and spring-driven vibrators.

As shown in FIG. 2, the gear shaft 38, the eccentric gear 40, and the orbital shaft 42 are positioned between the under fixing part 30 and the upper fixing part 32. The gear shaft 38 is surrounded by the lower bearing 34 and passes through the opening 54 in the under fixing part 30 and connects to the drive shaft 26 of the motor with a cusp gear 56 that inserts into the drive shaft 26. The eccentric gear 40 connects the gear shaft 38 to the orbital shaft 42, with an axis of the orbital shaft 42 offset from a rotation axis of the gear shaft 38. The amount of the offset can be selected to impart a desired movement or rotation of the mounting plate 22. The orbital shaft 42 is preferably surrounded by the upper bearing 36 and connected to a receiver in a bottom surface of the upper fixing part 32. As best shown in the partial section view of FIG. 3, the pair of o-rings 44 connect to hangers 58 on the under fixing part 30 and the upper fixing part 32 and serve to control the movement of the upper fixing part 32 relative to the under fixing part 30. In an alternative embodiment, the vibratory mechanism may include other gearing to provide alternative planar movement and/or vertical movement to the mounting plate 22 and tool 18.

In a preferred embodiment of this invention, the mounting plate 22 includes a releaseable connector 60 for attaching the tool 18. The releaseable connector 60 allows the grooming device 10 to utilize multiple tools 18 including, but not limited to, a nail file tool, emery boards, a buffing tool, a brush, a callous remover, a massage head and an exfoliator. In an embodiment, the tool 18 is connected to the mounting plate 22 with a hook and loop fastener. However, it should be understood that other types of releasable connectors may be used including, but not limited to, slot and tab connectors, screws, snap connectors, and clips. In an alternative embodiment, the removable tools may be adjustable, preferably pitch adjustable, providing different angles for operating on the nails. In a preferred embodiment, the grooming device 10 may utilize multiple emery boards with varying grit. For example, a rough grit may be used to shape a nail, a medium grit can be used to smooth ridges and surface irregularities, a fine-grained grit to buff and smooth the nail to a matte surface, and finally a finest finishing grit to give the nails a glossy shine for a professional look.

The embodiments of the invention described herein are presently preferred. Various modifications and improvements can be made without departing from the spirit and scope of the invention. The scope of the invention is defined by the appended claims, and all changes that fall within the meaning and range of equivalents are intended to be embraced therein.

We claim:

1. A grooming device comprising:
a handle including a motor and a drive shaft;
a swing head connectable to the handle, wherein the swing head includes an orbital vibratory mechanism with a mounting plate, the orbital vibratory mechanism connecting to the drive shaft to translate a rotation of the drive shaft to an orbital vibration of the mounting plate; and
a tool connectable to the mounting plate.

2. The grooming device of claim 1, wherein the tool comprises one of an emery board, a nail file, a nail polish remover, a brush, a callous remover, a massage head, and an exfoliator.

3. The grooming device of claim 2, wherein the tool is adjustable to change an angle of the tool relative to the mounting plate.

4. The grooming device of claim 1, wherein the tool is detachable and reattachable to the mounting plate.

5. The grooming device of claim 4, wherein the tool is replaceable with a second tool.

6. The grooming device of claim 1, wherein the handle is water resistant.

7. The grooming device of claim 1, further comprising a switch to connect the motor to a power supply to rotate the drive shaft.

8. The grooming device of claim 7, wherein the switch is further operable to change a rotation speed of the motor.

9. The grooming device of claim 1, wherein the vibratory mechanism comprises a gear shaft, an eccentric gear, and an orbital shaft, wherein the gear shaft connects to the drive shaft and an axis of the orbital shaft is offset from a rotation axis of the gear shaft and wherein rotating the rotation axis of the gear shaft imparts an orbital motion on the orbital shaft thereby imparting an orbital vibratory motion on the mounting plate.

10. A grooming device comprising:
a handle comprising an elongated body enclosing an interior;
a motor disposed in the interior of the handle and operative to drive a drive shaft extending out of the interior of the handle;
a swing head including an orbital vibratory mechanism with a mounting plate, the swing head connectable to the handle to engage the drive shaft with the orbital vibratory mechanism;
a switch operative to connect the motor to a power supply to rotate the drive shaft and to articulate the orbital vibratory mechanism and the mounting plate; and
a tool connectable to the mounting plate.

11. The grooming device of claim 10, wherein the comprises one of an emery board, a nail file, a nail polish remover, a brush, a callous remover, a massage head, and an exfoliator.

12. The grooming device of claim 11, wherein the tool is adjustable to change an angle of the tool relative to the mounting plate.

13. The grooming device of claim 10, wherein the tool is detachable and reattachable to the mounting plate.

14. The grooming device of claim 13, wherein the tool is replaceable with a second tool.

15. The grooming device of claim 10, wherein the handle is water resistant allowing the handle to be washed.

16. The grooming device of claim 10, wherein the switch is further operable to change a rotation speed of the motor.

17. The grooming device of claim 16, wherein the vibratory mechanism comprises a gear shaft connected to an eccentric gear connected to an orbital shaft, wherein the gear shaft connects to the drive shaft and an axis of the orbital shaft is offset from a rotation axis of the gear shaft and wherein rotating the gear shaft imparts an orbital motion on the orbital shaft thereby imparting an orbital vibratory motion on the mounting plate.

18. A grooming device comprising:
a handle including an opposed first end and second end and a hollow interior, wherein the interior houses a motor with a drive mechanism, the first end providing a connection to the drive mechanism and the second end providing access to the interior;
a swing head including a vibratory mechanism with a mounting plate, the swing head connected to the handle at the first end and engageable with the drive mechanism, wherein the vibratory mechanism includes a gear shaft connected to an eccentric gear connected to an orbital shaft, wherein the gear shaft connects to the drive mechanism and an axis of the orbital shaft is offset from a rotation axis of the gear shaft and wherein rotating the gear shaft imparts an orbital motion on the orbital shaft thereby imparting an orbital vibratory motion on the mounting plate;
a tool connected to the mounting plate, wherein the tool is detachable from the mounting plate and wherein the tool comprises one of a nail file, an emery board, a callous remover, an exfoliator, a massage head, and a brush.

19. The grooming device of claim 18, wherein the handle is water resistant thereby preventing damage to the motor when washing the handle.

20. The grooming device of claim 18, wherein the vibratory mechanism includes a pair of o-rings to control the motion of the vibratory mechanism.
